(19) United States
(12) Patent Application Publication
(10) Pub. No.: US 2007/0212401 A1
(43) Pub. Date: Sep. 13, 2007

(54) Single-use cosmetic article comprising a support impregnated with a lotion containing oxyethylenated sorbitan monolaurate comprising 4 OE

(76) Inventor: Virginie Masse, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/711,627

(22) Filed: Feb. 28, 2007

Related U.S. Application Data
(60) Provisional application No. 60/782,286, filed on Mar. 15, 2006.

(30) Foreign Application Priority Data
Feb. 28, 2006 (FR) 06 01757

(51) Int. Cl. A61K 9/70

(52) U.S. Cl. 424/443; 424/70.31

(57) ABSTRACT

The present disclosure relates to a single-use cosmetic article comprising at least one water-insoluble support impregnated with a lotion. The impregnating lotion comprises water, at least 1% by weight of at least one cosmetically acceptable alcohol and/or at least one cosmetically acceptable polyol, and at least 0.05% by weight of oxyethylenated sorbitan monolaurate comprising 4 OE. The disclosure also relates to a process for the cosmetic treatment of keratin materials such as the hair and/or the scalp, using such a cosmetic article.

This application claims benefit of U.S. Provisional Application No. 60/782,286, filed Mar. 15, 2006, the contents of which are incorporated herein by reference. This application also claims benefit of priority under 35 U.S.C. §119 to French Patent Application No. FR 06 01757, filed Feb. 28, 2006, the contents of which are also incorporated herein by reference.

The present disclosure relates to a single-use cosmetic article for use in caring for keratin materials, such as the hair or the scalp. More specifically, the present disclosure relates to a single-use cosmetic article comprising a water-insoluble support impregnated with a lotion which comprises, in an aqueous-alcoholic medium, a specific oxyalkylenated fatty acid ester of sorbitan.

The present disclosure also relates to a process for the cosmetic treatment of keratin materials using such a cosmetic article.

Compositions for washing keratin materials are generally in the form of more or less thick liquids. Thus, European Patent Application EP 0 115 888 describes a non-irritant composition for cleansing the skin, the scalp and the hair, which makes it possible to decrease the secretion of sebum related to frequent washing. This composition comprises from 5% to 10% by weight of non-ionic emulsifier having an HLB of between 10 and 19, and from 0.5% to 5% by weight of a thickener consisting of organic substances capable of forming gels and colloidal solutions in the presence of water.

European Patent Application EP 0 461 593 describes compositions for cleansing the hair, the detergent and cosmetic properties of which are improved. This composition comprises:
(a) from 0.05% to 15% by weight of a surfactant,
(b) from 0.5% to 40% by weight of an alcohol,
(c) from 0.1% to 25% by weight of water, and
(d) from 20% to 98% by weight of liquid oil,
   the ratio of the viscosity of the mixture comprising components (a), (b) and (c) to the viscosity of the oil (d) being greater than or equal to 1.

International Patent Application WO 03/096998 describes shampoo compositions that prevent the formation of dandruff. These compositions contain, as active agent, extracts of Camelia sinensis, or the active agents thereof such as polyphenols (catechins, flavonoids). These compositions can also contain additional anti-dandruff agents, and anionic, non-ionic, amphoteric, zwitterionic and/or cationic surfactants. Among non-ionic surfactants, mention is made of fatty acid esters of sorbitan, and polyoxyethylenated derivatives thereof comprising from 1 to 30, such as from 5 to 10 oxyethylene units.

Conventional compositions are liable, during use, to run into the eyes, and may cause discomforting reactions such as stinging or even irritation of the eyes.

Moreover, it is known practice to use cosmetic products in the form of a substrate impregnated with a cosmetic composition suitable for the desired purpose, in general removing makeup from and/or cleansing the skin and the eyes. These articles, commonly called “wipes,” generally meet with consumer approval due to their practical nature since they are disposable and impregnated with the required and sufficient amount of cosmetic product. The use of these wipes also avoids the handling and transport of boftles containing lotions or milks.

Thus, International Patent Application WO 01/01949 describes a personal cleansing composition, such as, for example, a makeup-removing composition, which has improved detergent properties while at the same time being non-irritant, in particular for the eyes and sensitive skin. This composition contains from 10% to 35% by weight of liquid silicone, from 10% to 35% by weight of a water-dispersible compound and from 55% to 65% by weight of an ester. This composition can be applied as it is to the skin, or via an impregnated support, for example a wipe.

U.S. Published Patent Application 2003/0119395 describes a disposable textile article, such as a wipe, which can be heated at least twice without chemical degradation. This article, intended for cleansing the skin, comprises a base layer impregnated with an aqueous solution which comprises a fragrance, at least one mild surfactant such as a mild anionic surfactant or amphoteric surfactant, at least one moisturizing agent and at least one preserving agent.

European Patent Application EP 1 405 634 describes an article of the dry wipe type, for cleansing and/or removing makeup from the skin and/or the eyes. This article comprises a substrate impregnated with a substantially anhydrous composition containing at least 10% by weight of one or more oils, at least one emulsifying surfactant and at least one hydrophilic gelling agent.

Finally, French Patent Application FR 2 867 066 describes a cosmetic hair composition which allows dry-cleansing of the hair. This composition comprises:
(a) from 10% to 90% by weight of at least one C1-C4 alcohol,
(b) from 0.01% to 5% by weight of at least one surfactant chosen from anionic, non-ionic, amphoteric or zwitterionic surfactants,
(c) from 0.01% to 5% by weight of an agent for solubilizing liposoluble compounds, and
(d) water.

This composition can be incorporated into a single-use cosmetic article consisting of a substrate impregnated with such a composition.

However, none of the disposable articles mentioned above necessarily makes it possible to render the cleansing of the keratin materials less aggressive. Moreover, none of these articles necessarily makes it possible to render the scalp less sensitive to outside attacks.

The present inventor has now discovered that, surprisingly, the incorporation, into a single-use cosmetic article such as, for example, a wipe, of a lotion comprising water, at least 1% by weight of an alcohol and/or a cosmetically acceptable polyol, and at least 0.05% by weight of a specific oxyalkylenated fatty acid ester of sorbitan makes it possible to facilitate, to improve and to make less aggressive the cleansing of keratin materials such as the hair and the scalp.

For instance, such an impregnated wipe makes it possible to prevent the cleansing product from running into the eyes.

In addition, and by virtue of its specific composition, the impregnating lotion is particularly mild and soothing for the scalp. The wipes according to the present disclosure thus make it possible, compared to the hair cleansing articles of the prior art, to reduce or prevent discomfort reactions of the scalp, such as redness, irritation or itching, caused by outside attacks and by the repeated use of more or less aggressive hair compositions.

The subject of the present disclosure is therefore a single-use cosmetic article comprising at least one water-insoluble support impregnated with a lotion comprising: water, at least 1% by weight of at least one cosmetically acceptable alcohol and/or at least one cosmetically acceptable polyol, and at least 0.05% by weight of oxyethylenated sorbitan monolaurate comprising 4 OE.

Another subject of the present disclosure is a process for the cosmetic treatment of keratin materials, such as the hair and the scalp, using such a cosmetic article.

Other subjects and characteristics, aspects and advantages of the present disclosure will emerge more clearly from reading the description and the examples which follow.

As used in the present disclosure, and in a manner well known per se, the term “compound comprising X OE” denotes an oxyethylenated compound comprising X oxyethylene units per molecule.

In addition, the term “Cy compound,” as used herein, denotes a compound containing Y carbon atoms.

According to the present disclosure, a support impregnated with a lotion (or “impregnating lotion”), which comprises water, at least one cosmetically acceptable alcohol and/or one cosmetically acceptable polyol, and oxyethylenated sorbitan monolaurate comprising 4 OE is used.

The expression “cosmetically acceptable alcohol and/or cosmetically acceptable polyol,” as used herein, denotes an alcohol and/or a polyol which is (are) compatible with keratin materials, such as the scalp, the skin and the hair.

The alcohols can, for instance, be chosen from C₁-C₄ lower alcohols, such as, for example, ethanol, isopropanol, tert-butanol and n-butanol, and mixtures thereof.

The polyols can, for example, be chosen from glycerol, propylene glycol and polyethylene glycols, and mixtures thereof.

In the context of the present disclosure, a mixture of alcohol(s) and of polyol(s) as described above can also be used.

The impregnating lotion may contain, for example, from 1% to 60% by weight of alcohol(s) and/or polyol(s), such as from 5% to 50% by weight, for example from 10% to 30% by weight, relative to the total weight of the lotion.

The oxyethylenated sorbitan monolaurate comprising 4 OE is, for example, sold under the name TWEEN 21 by the company Uniqema.

The impregnating lotion may also contain, in addition to the oxyethylenated sorbitan monolaurate comprising 4 OE, other esters of C₈-C₃₀ fatty acids and of oxyalkylenated sorbitan, comprising for example from 1 to 50 oxyalkylene units. Thus, according to an at least one embodiment, the impregnating lotion also contains oxyethylenated sorbitan monolaurate comprising 20 OE, such as, for example, the product sold under the name TWEEN 20 by the company Uniqema.

The impregnating lotion may contain, in at least one embodiment, from 0.05% to 10% by weight of oxyalkylenated fatty acid ester of sorbitan, such as from 0.1% to 8% by weight, for example from 0.2% to 5% by weight, relative to the total weight of the lotion.

According to at least one embodiment of the present disclosure, the impregnating lotion may also at least one additional surfactant chosen from anionic surfactants, cationic surfactants, amphoteric or zwitterionic surfactants, and non-ionic surfactants other than the oxyalkylenated fatty acid esters of sorbitan.

The anionic surfactants that can be used in said lotion are, in at least one embodiment, chosen from salts, e.g., alkali metal salts such as sodium salts, ammonium salts, amine salts, amino alcohol salts and alkaline-earth metal salts, for example magnesium salts, of the following types: alkyl sulphates, alkyl ether sulphates, alkylamido ether sulphates, alkylarylpolyether sulphates, monoglyceride sulphates, alkyl sulphonates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphoacetates, acyl sarcosinates and acyl glutamates, the alkyl and acyl groups of all these compounds containing from 6 to 24 carbon atoms and the aryl group being in at least one embodiment chosen from phenyl and benzyl groups.

It is also possible to use monoesters of C₆-C₂₄ alkyl and of polyglycoside-dicarboxylic acids, such as alkyl glucoside citrates, polyalkyl glycoside tartrates and polyalkyl glycoside sulphosuccinates, alkyl sulphosuccinamates, acyl isethionates and N-acyltaurates, the alkyl or acyl group of all these compounds containing from 12 to 20 carbon atoms.

Another group of anionic surfactants that can be used in the impregnating lotion is that of acyl lactylates in which the acyl group contains from 8 to 20 carbon atoms.

In addition, mention may also be made of alkyl-D-galactosiduronic acids and salts thereof, and also polyoxyalkylenated (C₆-C₂₄ alkyl) ether-carboxylic acids, polyoxyalkylenated (C₆-C₂₄ alkyl) (C₆-C₂₄ aryl) ether-carboxylic acids, polyoxyalkylenated (C₆-C₂₄ alkyl) amidoether-carboxylic acids and salts thereof, such as those comprising from 2 to 50 ethylene oxide units, and mixtures thereof.

According to one embodiment of the present disclosure, the anionic surfactant is chosen from alkyl sulphates, alkyl ether sulphates and mixtures thereof, such as in the form of alkali metal, alkaline-earth metal, ammonium, amine or amino alcohol salts.

When they are present, the amount of the anionic surfactant(s) can range, for example, from 0.01% to 50% by weight, such as from 0.1% to 20% by weight, relative to the total weight of the impregnating lotion.

Examples of non-ionic surfactants that can be used in the impregnating lotion according to the present disclosure are described, for example, in “Handbook of Surfactants” by M. R. Porter, published by Blackie & Son (Glasgow and London), 1991, pp. 116-178. They are chosen, for instance, from alcohols, alpha-diols, (C₁-C₂₀)alkylphenols or fatty acids which are polyethoxylated, polypropoxylated or polyglycerolated, having a fatty chain containing, for example, from 8 to 18 carbon atoms, it being possible for the number of ethylene oxide or propylene oxide groups to range, in at least one embodiment, from 2 to 50 and it being possible for the number of glycerol groups to range, in at least one embodiment, from 2 to 30.

Mention may also be made of condensates of ethylene oxide and of propylene oxide with fatty alcohols, polyethoxylated fatty amides having, for example, from 2 to 30 ethylene oxide units, polyglycerolated fatty amides comprising, on average, from 1 to 5 glycerol groups, such as from 1.5 to 4, fatty acid esters of sucrose, fatty acid esters of polyethylene glycol, (C₆-C₂₄ alkyl)polyglycosides, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

When they are present, the amount of the non-ionic surfactant(s) can range, for example, from 0.01% to 20% by weight, such as from 0.1% to 10% by weight, such as from 0.1% to 5% by weight, relative to the total weight of the impregnating lotion.

The amphoteric or zwitterionic surfactants that may be incorporated into the impregnating lotion can, in at least one embodiment, be derivatives of secondary or tertiary aliphatic amines, in which the aliphatic group is a linear or branched chain comprising from 8 to 22 carbon atoms and containing at least one anionic group such as, for example, a carboxylate, sulphonate, sulphate, phosphate or phosphonate group. Mention may, for example, be made of (C₈-C₂₀)alkylbetaines, sulphobetaines, (C₈-C₂₀ alkyl)amido(C₆-C₈ alkyl)betaines or (C₈-C₂₀ alkyl)amido(C₆-C₈ alkyl)sulphobetaines.

Among the amine derivatives, mention may be made of the products sold under the name Miranol®, as described in U.S. Pat. Nos. 2,528,378 and 2,781,354 and classified in the CTFA dictionary, 3rd edition, 1982, under the names Amphocarboxyglycinate and Amphocarboxypropionate having the respective structures (II) and (III):
Rₐ—CONHCH₂CH₂—N(R_{b})(R_{c})(CH₂COO⁻) (II)
in which:
Rₐ is chosen from an alkyl group derived from an acid Rₐ—COOH present in hydrolyzed coconut oil, and from a heptyl group, a nonyl group, and an undecyl group,
R_{b} is a beta-hydroxyethyl group, and
R_{c} is a carboxymethyl group;
and
   Rₐ′—CONHCH₂CH₂—N(B)(B′) (III)
in which:
B is —CH₂CH₂OX′,
B′ is —(CH₂)_{z}—Y′, with z=1 or 2,
X′ is chosen from the group —CH₂CH₂—COOH and a hydrogen atom,
Y′ is chosen from —COOH and the group —CH₂—CHOH—SO₃H,
Rₐ′ is chosen from an alkyl group of an acid Rₐ′—COOH present in coconut oil or in hydrolyzed linseed oil, an alkyl group, such as a C₁₇ alkyl group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M concentrate.

Among the amphoteric or zwitterionic surfactants mentioned above, use is made in at least one embodiment of (C₈-C₂₀ alkyl)betaines, (C₈-C₂₀ alkyl)amido(C₆-C₈ alkyl)betaines, and mixtures thereof.

When they are present, the amount of the amphoteric or zwitterionic surfactant(s) can range, for example, from 0.01% to 20% by weight, such as from 0.05% to 10% by weight, for example from 0.1% to 2% by weight, relative to the total weight of the impregnating lotion.

By way of examples of cationic surfactants that may be part of the composition of the impregnating lotion, mention may be made, for example, of the salts of primary, secondary or tertiary fatty amines, which are optionally polyoxyalkylenated; quaternary ammonium salts, such as tetraalkylammonium, alkylamidoalkyltrialkylammonium, trialkylbenzylammonium, trialkylhydroxyalkylammonium or alkylpyridinium chlorides or bromides; imidazoline derivatives.

When they are present, the amount of the cationic surfactant(s) can range, for example, from 0.01% to 10% by weight, such as from 0.2% to 5% by weight, for example from 0.3% to 3% by weight, relative to the total weight of the impregnating lotion.

According to a first embodiment of the present disclosure, the total amount of surfactants is within the range of from 0.01% to 50% by weight, for example from 0.05% to 20% by weight, or from 0.1% to 10% by weight, relative to the total weight of the impregnating lotion.

According to a second embodiment of the present disclosure, the impregnating lotion contains at least one anionic surfactant and/or at least one amphoteric or zwitterionic surfactant.

According to another embodiment of the present disclosure, the impregnating lotion can also contain at least one fatty substance. The fatty substances that can be used in the present disclosure may, for example, be chosen from plant oils, animal oils, mineral oils, natural or synthetic oils, fatty alcohols, linear or branched, volatile or non-volatile silicones which may or may not be organomodified, and mixtures of such fatty substances.

As plant oil, mention may, for example, be made of sweet almond oil, avocado oil, castor oil, olive oil, liquid jojoba wax, sunflower oil, wheat germ oil, sesame oil, groundnut oil, grapeseed oil, soybean oil, rapeseed oil, safflower oil, coconut oil, maize oil, hazelnut oil, palm oil, apricot kernel oil, beauty-leaf oil, evening primrose oil, shea butter, rice bran oil, maize germ oil, passion flower oil and rye oil.

As animal oil, mention may, for example, be made of perhydrosqualene.

As mineral oil, mention may, for instance, be made of paraffin oils and liquid petroleum jelly.

As synthetic oil, mention may, for instance, be made of squalane, poly(α-olefins) such as isododecane or isohexadecane, transesterified plant oils and fluoro oils, and fatty esters.

The term “fatty esters,” as used herein, denotes compounds of formula RₐCOOR_{b} in which Rₐ represents the residue of a hydroxylated or non-hydroxylated, saturated or unsaturated, linear or branched higher acid containing from 4 to 29 carbon atoms, and R_{b} represents a linear or branched, saturated or unsaturated hydrocarbon-based chain containing from 3 to 30 carbon atoms, the total number of carbon atoms of the ester being greater than 10. By way of examples, mention may be made of Purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate or tridecyl neopentanoate.

The fatty alcohols include, for example, and non limitatively, myristyl alcohol, cetyl alcohol, stearyl alcohol, arachidyl alcohol, behenyl alcohol and erucyl alcohol.

The silicones can be, for example, polydimethylsiloxanes and aminated polydimethylsiloxanes, in the form of oils, gums or resins.

According to at least one embodiment of the present disclosure, the fatty substances as described above are present in an amount ranging from 0.01% to 20% by weight, such as from 0.1% to 10% by weight, for example from 0.5% to 5% by weight, relative to the total weight of the impregnating lotion.

According to another embodiment of the present disclosure, the impregnating lotion can also comprises at least one anti-dandruff agent. As anti-dandruff agents, use may be made, for example, of compounds such as piroctone olamine, zinc pyrithione, salicylic acid, selenium disulphide, and mixtures thereof.

The impregnating lotion may then contain from 0.001% to 10% by weight of the at least one anti-dandruff agent, for example from 0.1% to 5% by weight, such as from 0.2% to 2% by weight, relative to the total weight of said lotion.

The impregnating lotion may also comprise at least one conventional additive well known in the art, such as calmatives, agents for preventing hair loss, anti-ageing agents, oxidizing agents, vitamins and provitamins, including panthenol, ceramides and pseudoceramides, sunscreens, colored or colorless, inorganic or organic pigments, dyes, pearlescent agents and opacifiers, sequestering agents, plasticizers, solubilizing agents, acidifying agents, basifying agents, inorganic or organic thickeners, antioxidants, hydroxy acids, fragrances and preserving agents.

Those skilled in the art will take care to select the optional additives and the amounts thereof in such a way that they do not harm the properties of the cosmetic articles which are the subjects of the present disclosure.

These additives may be present in the impregnating lotion in an amount ranging from 0 to 20% for each by weight relative to the total weight of said lotion.

The support that is part of the composition of the cosmetic articles according to the disclosure is water-insoluble.

This support can comprise one or more layers which can be chosen from woven materials, nonwoven materials, foams, sponges and cotton wool, it being possible for these materials to be, for example, in the form of sheets, balls or films.

According to one embodiment of the present disclosure, the support comprises at least one layer comprising a nonwoven material. Such a material may be based on fibers of natural origin (flax, wool, cotton, silk) or of synthetic origin (cellulose derivatives, viscose, polyvinyl derivatives, polyesters such as polyethylene terephthalate, polyolefins such as polyethylene or polypropylene, polyamides such as nylon, acrylic derivatives). Nonwoven materials are described in general in Riedel “Nonwoven Bonding Methods & Materials”, Nonwoven World (1987). These materials are obtained according to the usual processes of the technique for preparing nonwoven materials.

When the support is a nonwoven material, a sufficiently thick nonwoven material may be used, which does not roll up into a ball and which is solid enough not to disintegrate and not to pill when applied to the scalp. It should be absorbent, and may be, in at least one embodiment, soft on at least one face.

As suitable nonwoven materials, mention may, for example, be made of those sold under the names ULTRALOFT 15285-01, ULTRALOFT 182-008, ULTRALOFT 182-010 and ULTRALOFT 182-016 by the company BBA, VILMED M1519 BLAU, VILMED M 1550 and 112-132-3 by the company Freudenberg, that sold under the name NORAFIN 11601-010B by the company Jacob Holm Industries, the flocked nonwovens sold under the names UNIVEL 109 and UNIVEL 119 by the company Uni Flockage, and the nonwoven materials sold under the name SAWATEX by the company Sandler.

According to another embodiment of the present disclosure, the support may comprise one or more layers that may have identical or different properties and may have elasticity and softness properties and other properties suitable for the desired use. The supports may comprise, for example, two parts that have different elasticity properties, as described in International Patent Application WO-A-99/13861, or comprise a single layer with different densities, as described in International Patent Application WO-A-99/25318, or comprise two layers of different textures, as described in International Patent Application WO-A-98/18441.

According to one embodiment of the present disclosure, the support comprises at least one layer having a weight within the range of from 30 to 90 g/m², such as, for example, from 40 to 70 g/m².

The support may have any size and any form suitable for the desired purpose. It may thus have, for example, the form of a rectangular wipe, or the form of a glove or of a mitten, easy to slip onto the hand, or the form of a round compress. It may have a surface area ranging from 0.005 m² to 0.1 m², such as, for example, from 0.01 m² to 0.05 m².

The degree of impregnation by weight of the support, with the lotion, may range from 10% to 1500%, such as from 50% to 500%, for example from 100% to 400%.

The techniques for impregnating a support with a lotion are well known in this field, and are all applicable to the present disclosure. In general, the impregnating lotion is added to the support by means of one or more techniques comprising immersion, coating, spraying, etc.

Another subject of the present disclosure is also a process for the cosmetic treatment of keratin materials, for example a process for the cosmetic cleansing or care of keratin materials, comprising passing a cosmetic article as described above over said materials, and then optionally rinsing after an optional leave-in time.

According to one embodiment, the cosmetic articles according to the disclosure are used in a non-rinse-off mode, i.e., the above process does not comprise a rinsing step. This embodiment is advantageous in so far as it makes it possible to definitely avoid any contact of the impregnating lotion with the eyes.

In at least one embodiment, the keratin materials are the hair and/or the scalp.

The present disclosure also relates to the use of a cosmetic article as defined above, for cleansing or caring for keratin materials, such as the hair and/or the scalp.

Finally, the present disclosure relates to a method for reducing discomfort reactions of the scalp, comprising passing a cosmetic article as described above over the keratin materials, for example the scalp, and then optionally rinsing after an optional leave-in time.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following example is given by way of illustration of the present disclosure, and can in no way limit the scope.

### EXAMPLE

Cosmetic articles in accordance with the invention, comprising the lotions A and B having a composition given in detail in the table below were prepared.

In the table hereinafter, all the amounts are indicated as percentage by weight of active material relative to the total weight of the lotion.

| Lotion composition | A | B |
|---|---|---|
| Ethyl alcohol at 96 degrees | 15.8 | 15.8 |
| Mixture of isoparaffin-based hydrocarbons ⁽¹⁾ | 1 | 1 |
| Oxyethylenated sorbitan monolaurate (4 OE) ⁽²⁾ | 0.25 | 0.5 |
| Sodium N-cocoylamidoethyl-N-ethoxycarboxymethyl- | 0.2 | — |
| glycinate ⁽³⁾ | | |
| Methyl p-hydroxybenzoate | 0.2 | 0.2 |
| Fragrance | 0.1 | 0.1 |
| Water qs | 100% | 100% |

⁽¹⁾ sold under the name ISOPAR H by the company EXXON MOBIL,
⁽²⁾ sold under the name TWEEN 21 by the company UNIQEMA,
⁽³⁾ sold under the name MIRANOL C2M by the company RHODIA CHIMIE.

Wipes were prepared from each of these lotions, by impregnating a support made of a nonwoven, sold under the name SAWATEX 2647 by the company SANDLER, with a degree of impregnation by weight of the support, with the lotion, of 250%. This support has a weight of approximately 55 g/m², comprises viscose and polypropylene fibers, and was used in glove form.

The performance levels of the wipes described above were evaluated by professionals, on panels of models, by applying the wipe to dry hair and the scalp, massaging slightly.

After application, the hair dried very rapidly. It was clean and soft, and the scalp was not irritated.

What is claimed is:

1. A single-use cosmetic article comprising at least one water-insoluble support impregnated with a lotion comprising: water, at least 1% by weight of at least one cosmetically acceptable alcohol and/or at least one cosmetically acceptable polyol, and at least 0.05% by weight of oxyethylenated sorbitan monolaurate comprising 4 OE, relative to the total weight of the lotion.

2. A cosmetic article according to claim 1, wherein the impregnating lotion comprises at least one alcohol chosen from C₁-C₄ lower alcohols.

3. A cosmetic article according to claim 1, wherein the impregnating lotion comprises at least one polyol chosen from glycerol, propylene glycol, polyethylene glycols, and mixtures thereof.

4. A cosmetic article according to claim 1, wherein the impregnating lotion comprises from 1% to 60% by weight of said at least one alcohol and/or said at least one polyol, relative to the total weight of the lotion.

5. A cosmetic article according to claim 4, wherein the impregnating lotion comprises from 5% to 50% by weight of said at least one alcohol and/or said at least one polyol, relative to the total weight of the lotion.

6. A cosmetic article according to claim 1, wherein the impregnating lotion further comprises oxyethylenated sorbitan monolaurate comprising 20 OE.

7. A cosmetic article according to claim 1, wherein the impregnating lotion further comprises from 0.05% to 10% by weight of an oxyalkylenated fatty acid ester of sorbitan, other than said oxyethylenated sorbitan monolaurate comprising 4 OE, relative to the total weight of the lotion.

8. A cosmetic article according to claim 7, wherein the impregnating lotion comprises from 0.1% to 8% by weight of oxyalkylenated fatty acid ester of sorbitan, relative to the total weight of the lotion.

9. A cosmetic article according to claim 8, wherein the impregnating lotion comprises from 0.2% to 5% by weight of oxyalkylenated fatty acid ester of sorbitan, relative to the total weight of the lotion.

10. A cosmetic article according to claim 1, wherein the impregnating lotion comprises at least one additional surfactant chosen from anionic surfactants, cationic surfactants, amphoteric or zwitterionic surfactants, and non-ionic surfactants other than the oxyalkylenated fatty acid esters of sorbitan.

11. A cosmetic article according to claim 10, wherein the impregnating lotion comprises at least one anionic surfactant chosen from salts of the following types: alkyl sulphates, alkyl ether sulphates, alkylamido ether sulphates, alkylarylpolyether sulphates, monoglyceride sulphates, alkyl sulphonates, alkylamide sulphonates, alkylaryl sulphonates, a-olefin sulphonates, paraffin sulphonates, alkyl sulphosuccinates, alkyl ether sulphosuccinates, alkylamide sulphosuccinates, alkyl sulphoacetates, acyl sarcosinates and acyl glutamates, the alkyl and acyl groups of all these compounds comprising from 6 to 24 carbon atoms.

12. A cosmetic article according to claim 11, wherein said salts are chosen from alkali metal salts, ammonium salts, amine salts, amino alcohol salts and alkaline-earth metal salts.

13. A cosmetic article according to claim 10, wherein the aryl groups of all the salts are chosen from phenyl and benzyl groups.

14. A cosmetic article according to claim 10, wherein the impregnating lotion comprises at least one anionic surfactant chosen from monoesters of C₆-C₂₄ alkyl and of polyglycoside-dicarboxylic acids, the alkyl or acyl group of these compounds comprising from 12 to 20 carbon atoms.

15. A cosmetic article according to claim 14, wherein said monoesters of C₆-C₂₄ alkyl and of polyglycoside-dicarboxylic acids are chosen from alkyl glucoside citrates, polyalkyl glycoside tartrates and polyalkyl glycoside sulphosuccinates, alkyl sulpho-succinamates, acyl isethionates and N-acyltaurates.

16. A cosmetic article according to claim 10, wherein the impregnating lotion comprises at least one anionic surfactant chosen from acyl lactylates in which the acyl group contains from 8 to 20 carbon atoms, alkyl-D-galactosiduronic acids and salts thereof, and also polyoxyalkylenated (C₆-C₂₄ alkyl) ether-carboxylic acids, polyoxyalkylenated (C₆-C₂₄ alkyl) (C₆-C₂₄ aryl) ether-carboxylic acids, polyoxyalkylenated (C₆-C₂₄ alkyl) amidoether-carboxylic acids and salts thereof.

17. A cosmetic article according to claim 10, wherein the at least one anionic surfactant is are present in an amount ranging from 0.01% to 50% by weight, relative to the total weight of the impregnating lotion.

18. A cosmetic article according to claim 17, wherein the at least one anionic surfactant is present in an amount ranging from 0.1% to 20% by weight, relative to the total weight of the impregnating lotion.

19. A cosmetic article according to claim 10, wherein the impregnating lotion comprises at least one amphoteric or zwitterionic surfactant chosen from derivatives of secondary or tertiary aliphatic amines in which the aliphatic group is a linear or branched chain comprising from 8 to 22 carbon atoms and comprising at least one anionic group.

20. A cosmetic article according to claim 19, wherein said at least one anionic group is chosen from carboxylate, sulphonate, sulphate, phosphate and phosphonate groups.

21. A cosmetic article according to claim 10, wherein the impregnating lotion comprises at least one amphoteric or zwitterionic surfactant chosen from (C₈-C₂₀)alkylbetaines, sulphobetaines, (C₈-C₂₀ alkyl)amido(C₆-C₈ alkyl)betaines and (C₈-C₂₀ alkyl)amido(C₆-C₈ alkyl)sulphobetaines.

22. A cosmetic article according to claim 19, wherein the at least one amphoteric or zwifterionic surfactant is present in an amount ranging from 0.01% to 20% by weight, relative to the total weight of the impregnating lotion.

23. A cosmetic article according to claim 22, wherein the at least one amphoteric or zwitterionic surfactant is present in an amount ranging from 0.05% to 10% by weight, relative to the total weight of the impregnating lotion.

24. A cosmetic article according to claim 10, wherein the impregnating lotion comprises at least one cationic surfactant chosen from salts of primary, secondary or tertiary fatty amines, which are optionally polyoxyalkylenated; quaternary ammonium salts; and imidazoline derivatives.

25. A cosmetic article according to claim 24, wherein the at least one cationic surfactant is present in an amount ranging from 0.01% to 10% by weight, relative to the total weight of the impregnating lotion.

26. A cosmetic article according to claim 10, wherein the impregnating lotion comprises at least one non-ionic surfactant chosen from alcohols, alpha-diols, (C₁-C₂₀)alkylphenols and fatty acids which are polyethoxylated, polypropoxylated or polyglycerolated, condensates of ethylene oxide and of propylene oxide with fatty alcohols, polyethoxylated fatty amides, polyglycerolated fatty amides comprising, on average, from 1 to 5 glycerol groups, fatty acid esters of sucrose, fatty acid esters of polyethylene glycol, (C₆-C₂₄ alkyl)polyglycosides, N—(C₆-C₂₄ alkyl)glucamine derivatives, and amine oxides.

27. A cosmetic article according to claim 26, wherein said amine oxides are chosen from (C₁₀-C₁₄ alkyl)amine oxides and N—(C₁₀-C₁₄ acyl)aminopropylmorphonine oxides.

28. A cosmetic article according to claim 26, wherein the at least one non-ionic surfactant is present in an amount ranging from 0.01% to 20% by weight, relative to the total weight of the impregnating lotion.

29. A cosmetic article according to claim 28, wherein the at least one non-ionic surfactant is present in an amount ranging from 0.1% to 10% by weight, relative to the total weight of the impregnating lotion.

30. A cosmetic article according to claim 10, wherein the total amount of surfactants ranges from 0.01% to 50% by weight, relative to the total weight of the impregnating lotion.

31. A cosmetic article according to claim 30, wherein the total amount of surfactants ranges from 0.05% to 20% by weight, relative to the total weight of the impregnating lotion.

32. A cosmetic article according to claim 1, wherein the impregnating lotion also comprises at least one fatty substance chosen from plant oils, animal oils, mineral oils, natural or synthetic oils, fatty alcohols, linear or branched, volatile or non-volatile silicone oils which may or may not be organomodified, and mixtures of such fatty substances.

33. A cosmetic article according to claim 32, wherein the impregnating lotion comprises from 0.01% to 20% by weight of said at least one fatty substance, relative to the total weight of the impregnating lotion.

34. A cosmetic article according to claim 33, wherein the impregnating lotion comprises from 0.1% to 10% by weight of said at least one fatty substance, relative to the total weight of the impregnating lotion.

35. A cosmetic article according to claim 1, wherein the impregnating lotion also comprises from 0.001% to 10% by weight of at least one anti-dandruff agent.

36. A cosmetic article according to claim 1, wherein the impregnating lotion also comprises at least one conventional additive chosen from calmatives, agents for preventing hair loss, anti-ageing agents, oxidizing agents, vitamins and provitamins, ceramides and pseudoceramides, sunscreens, colored or colorless, inorganic or organic pigments, dyes, pearlescent agents and opacifiers, sequestering agents, plasticizers, solubilizing agents, acidifying agents, basifying agents, inorganic or organic thickeners, antioxidants, hydroxy acids, fragrances and preserving agents.

37. A cosmetic article according to claim 1, wherein the at least one water-insoluble support comprises at least one layer chosen from woven materials, nonwoven materials, foams, sponges and cotton wool.

38. A cosmetic article according to the claim 37, wherein the at least one water-insoluble support comprises at least one layer of a nonwoven material, comprising fibers of natural origin or of synthetic origin.

39. A cosmetic article according to claim 1, wherein the at least one water-insoluble support comprises at least one layer having a weight ranging from 30 to 90 g/m².

40. A cosmetic article according to claim 39, wherein the at least one water-insoluble support comprises at least one layer having a weight ranging from 40 to 70 g/m².

41. A cosmetic article according to claim 1, wherein the at least one water-insoluble support has a form chosen from a rectangular wipe, a glove or a mitten, and a round compress.

42. A cosmetic article according to claim 1, wherein the at least one water-insoluble support has a surface area ranging from 0.005 m² to 0.1 m².

43. A cosmetic article acording to claim 42, wherein said at least one water-insoluble support has a surface area ranging from 0.01 m² to 0.05 m².

44. A cosmetic article according to claim 1, wherein the degree of impregnation by weight of the support, with the lotion, ranges from 10% to 1500%.

45. A cosmetic article according to claim 44, wherein the degree of impregnation by weight of the support, with the lotion, ranges from 50% to 500%.

46. A process for the cosmetic treatment of keratin materials, comprising passing a cosmetic article over said materials, and then optionally rinsing after an optional leave-in time, wherein said cosmetic article is a single-use cosmetic article comprising at least one water-insoluble support impregnated with a lotion comprising: water, at least 1% by weight of at least one cosmetically acceptable alcohol and/or at least one cosmetically acceptable polyol, and at least 0.05% by weight of oxyethylenated sorbitan monolaurate comprising 4 OE, relative to the total weight of the lotion.

47. A process according to claim 46, wherein said process does not comprise a rinsing step.

48. A process for cleansing or caring for keratin materials, comprising passing a cosmetic article over said materials, and then optionally rinsing after an optional leave-in time, wherein said cosmetic article is a single-use cosmetic article comprising at least one water-insoluble support impregnated with a lotion comprising: water, at least 1% by weight of at least one cosmetically acceptable alcohol and/or at least one cosmetically acceptable polyol, and at least 0.05% by weight of oxyethylenated sorbitan monolaurate comprising 4 OE, relative to the total weight of the lotion.

49. A process for cleansing or caring for keratin materials according to claim 48, wherein said keratin materials are chosen from the hair and the scalp.

50. A method for reducing discomfort reactions of the scalp, comprising passing a cosmetic article over the scalp, and then optionally rinsing after an optional leave-in time, wherein said cosmetic article is a single-use cosmetic article comprising at least one water-insoluble support impregnated with a lotion comprising: water, at least 1% by weight of at least one cosmetically acceptable alcohol and/or at least one cosmetically acceptable polyol, and at least 0.05% by weight of oxyethylenated sorbitan monolaurate comprising 4 OE, relative to the total weight of the lotion.
